(19) United States
(12) Patent Application Publication
(10) Pub. No.: US 2026/0045318 A1
(43) Pub. Date: Feb. 12, 2026

(54) ALTERNATIVE PROTEIN MATERIAL PREDICTION DEVICE AND METHOD

(71) Applicant: Kookmin University Industry Academy Cooperation Foundation, Seoul (KR)

(72) Inventors: Hee YANG, Seoul (KR); Eunyeong LEE, Seoul (KR)

(73) Assignee: Kookmin University Industry Academy Cooperation Foundation, Seoul (KR)

(21) Appl. No.: 19/263,082

(22) Filed: Jul. 8, 2025

(30) Foreign Application Priority Data
Aug. 8, 2024 (KR) 10-2024-0106360

(51) Int. Cl. G16B 15/20; G16B 40/20

(52) U.S. Cl. CPC G16B 15/20; G16B 40/20

(57) ABSTRACT

The present disclosure relates to an alternative protein material prediction device. The device includes a protein feature extractor configured to composite features of protein as sequence features, structural features, and physicochemical features; a protein graph data generator configured to generate nodes based on the sequence features and physicochemical features of the protein and generate edges between the nodes based on the structural features of the protein, thereby generating protein graph network data, and an alternative material protein predictor configured to generate an alternative protein material prediction model for predicting an alternative protein material by learning the protein graph data that reflects the composite features of the protein.

### CROSS-REFERENCE TO RELATED APPLICATION

This application claims the benefit of and priority to Korean Patent Application No. 10-2024-0106360, filed on Aug. 8, 2024, the entire disclosure(s) of which is hereby incorporated herein by reference in its entirety.

### TECHNICAL FIELD

The present disclosure relates to an alternative protein material prediction technology, and more specifically, to an alternative protein material prediction device and method capable of predicting an alternative protein material that mimics the functional features of conventional animal-based food using an artificial intelligence model trained by converting composite features of protein into protein graph data.

### BACKGROUND

As the demand for alternative proteins increases due to issues such as food security and environmental pollution, it is important to understand the features and functions of plant-based, microbial-based, or synthetic proteins that can substitute conventional animal-based protein sources and to identify suitable raw materials. However, the method of searching new plant-based materials with excellent functional features is time-consuming, costly, and labor-intensive. Therefore, the application of efficient screening and prediction technology is required.

As a similar case, Korean Patent No. 10-2617958 (Dec. 20, 2023) relates to a method and device for predicting compound-protein interactions based on a cross-attention mechanism. The method for predicting the compound-protein interactions based on the cross-attention mechanism may include a step of encoding compound information based on molecular graph data and molecular fingerprint data, a step of encoding protein information based on protein sequence data, a step of inputting the encoded compound information and protein information into a first cross-attention block, and a step of predicting an interaction between the compound and the protein based on the output of the first cross-attention block.

The main field of alternative protein prediction may include understanding and selection of protein materials, analysis of functional and structural features, bioinformatics and computer-based prediction models, development and optimization of alternative proteins, and regulatory and safety assessments, plays an important role in the discovery and development of sustainable food materials, and is evaluated as a key element in promoting innovation in the food industry.

### PRIOR ART DOCUMENT

(Patent Document) 10-2617958 (Dec. 20, 2023)

### SUMMARY

In view of the above, the present disclosure provides an alternative protein material prediction device and method capable of predicting an alternative protein material that mimics the functional features of conventional animal-based food using an artificial intelligence model trained by converting composite features of protein into protein graph data.

The present disclosure provides an alternative protein material prediction device and method that generates protein graph data capable of simultaneously considering sequence features, physicochemical features, and structural features of protein, thereby comprehensively learning protein features.

The present disclosure provides an alternative protein material prediction device and method that can extract composite features of protein, generate protein graph data, and perform an alternative protein material prediction procedure, as the role of performing an alternative protein material prediction service.

The present disclosure provides an alternative protein material prediction device including a protein feature extractor configured to extract composite features of protein as sequence features, structural features, and physicochemical features, a protein graph data generator configured to generate nodes based on the sequence features and physicochemical features of the protein and generate edges between the nodes based on the structural features of the protein, thereby generating protein graph data, and an alternative material protein predictor configured to generate an alternative protein material prediction model for predicting an alternative protein material by learning the protein graph data that reflects the composite features of the protein.

The protein feature extractor may determine the sequence features, structural features, and physicochemical features of the protein through a protein information database that stores composite features of animal and plant proteins.

The protein feature extractor may extract the sequence features of the protein through a language model. The protein feature extractor may extract the physicochemical features of the protein using a protein descriptor comprising at least CTD (Composition, Transition, Distribution) or PseAAC (Pseudo amino acid composition). The protein feature extractor may extract the structural features of the protein using a graph network data processing technique on the protein's 3D structural data.

The protein graph data generator may assign a protein characterization code to the node based on a language representing the sequence features and amino acid feature values representing the physicochemical features. The protein graph data generator may assign weight codes of the edges based on amino acid interactions or spatial proximity that represent the structural features. The protein graph data generator may generate the sequence features, the physicochemical features, and the structural features all at once as the protein graph data.

The alternative material protein predictor may determine at least one alternative protein for the protein based on the predicted protein graph data. The alternative material protein predictor may compare composite features for at least one alternative protein with the composite features of the protein to predict the functional features of food and recommend an optimal alternative protein.

The alternative material protein predictor may determine plant protein capable of substituting the animal protein by inputting the composite features of animal protein into the alternative protein material prediction model.

The present disclosure provides an alternative protein material prediction method that may be performed by a computing device, the method including a protein feature extraction step of extracting composite features of protein as sequence features, structural features, and physicochemical features, a protein graph data generation step of generating nodes based on the sequence features and physicochemical features of the protein and generating edges between the nodes based on the structural features of the protein, thereby generating protein graph data, and an alternative material protein prediction step of generating an alternative protein material prediction model for predicting the protein graph data by learning the composite features of the protein.

The protein feature extraction step may include a step of determining the sequence features, structural features, and physicochemical features of the protein through a protein information database that stores composite features of animal and plant proteins.

The protein graph data generation step may include a step of assigning a protein characterization code to the node based on a language representing the sequence features and amino acid feature values representing the physicochemical features.

The alternative material protein prediction step may include a step of determining at least one alternative protein for the protein based on the predicted protein graph data.

The alternative material protein prediction step may include a step of determining plant protein capable of substituting the animal protein by inputting the composite features of animal protein into the alternative protein material prediction model.

### Advantageous Effects

The disclosed technology may have the following effects. However, since this does not mean that a specific embodiment should include all or only the following effects, the scope of the disclosed technology should not be construed as being limited thereto.

An alternative protein material prediction device and method according to an embodiment of the present disclosure can predict an alternative protein material that mimics the functional features of conventional animal-based food using an artificial intelligence model trained by converting composite features of protein into protein graph data.

An alternative protein material prediction device and method according to an embodiment of the present disclosure can generate sequence features, physicochemical features, and structural features all at once as protein graph data.

An alternative protein material prediction device and method according to an embodiment of the present disclosure can extract composite features of protein, generate protein graph data, and perform an alternative protein material prediction procedure, as the role of performing an alternative protein material prediction service.

### BRIEF DESCRIPTION OF THE DRAWINGS

FIG. 1 is a diagram illustrating an alternative protein material prediction system according to an embodiment of the present disclosure.

FIG. 2 is a diagram illustrating the configuration of the alternative protein material prediction device of FIG. 1.

FIG. 3 is a diagram illustrating the functional configuration of the alternative protein material prediction device of FIG. 1.

FIG. 4 is a flowchart showing the operational process of an alternative protein material prediction device 130.

FIG. 5 is a flowchart illustrating the operation of the alternative protein material prediction device shown in FIG. 3.

FIG. 6 is a diagram illustrating the process of selecting an alternative protein source based on the similarity of features of each raw material.

### DETAILED DESCRIPTION

The explanation of the present disclosure is merely an embodiment for structural or functional explanation, so the scope of the present disclosure should not be construed to be limited to the embodiments explained in the embodiment. That is, since the embodiments may be implemented in several forms without departing from the characteristics thereof, it should also be understood that the described embodiments are not limited by any of the details of the foregoing description, unless otherwise specified, but rather should be construed broadly within its scope as defined in the appended claims. Therefore, various changes and modifications that fall within the scope of the claims, or equivalents of such scope are therefore intended to be embraced by the appended claims.

Terms described in the present disclosure may be understood as follows.

While terms such as “first,” “second,” etc., may be used to describe various components, such components must not be understood as being limited to the above terms. The above terms are used to distinguish one component from another. For example, a first component may be referred to as a second component without departing from the scope of rights of the present disclosure, and likewise a second component may be referred to as a first component.

It will be understood that when an element is referred to as being “connected to” another element, it may be directly connected to the other element or intervening elements may also be present. In contrast, when an element is referred to as being “directly connected to” another element, no intervening elements are present. In addition, unless explicitly described to the contrary, the word “comprise” and variations such as “comprises” or “comprising,” will be understood to imply the inclusion of stated elements but not the exclusion of any other elements. Meanwhile, other expressions describing relationships between components such as “between,” “immediately between” or “adjacent to” and “directly adjacent to” may be construed similarly.

Singular forms “a,” “an” and “the” in the present disclosure are intended to include the plural forms as well, unless the context clearly indicates otherwise. It will be further understood that terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

In each phase, reference numerals (for example, a, b, c, etc.) are used for the sake of convenience in description, and such reference numerals do not describe the order of each phase. The order of each phase may vary from the specified order, unless the context clearly indicates a specific order. In other words, each phase may take place in the same order as the specified order, may be performed substantially simultaneously, or may be performed in a reverse order.

The present disclosure may be implemented as machine-readable codes on a machine-readable medium. The machine-readable medium may include any type of recording device for storing machine-readable data. Examples of the machine-readable recording medium may include a read-only memory (ROM), a random access memory (RAM), a compact disk-read only memory (CD-ROM), a magnetic tape, a floppy disk, optical data storage, or any other appropriate type of machine-readable recording medium. The medium may also be carrier waves (for example, Internet transmission). The computer-readable recording medium may be distributed among networked machine systems which store and execute machine-readable codes in a de-centralized manner.

The terms used in the present application are merely used to describe particular embodiments, and are not intended to limit the present disclosure. Unless otherwise defined, all terms used herein, including technical or scientific terms, have the same meanings as those generally understood by those with ordinary knowledge in the field of art to which the present disclosure belongs. Such terms as those defined in a generally used dictionary are to be interpreted to have the meanings equal to the contextual meanings in the relevant field of art, and are not to be interpreted to have ideal or excessively formal meanings unless clearly defined in the present application.

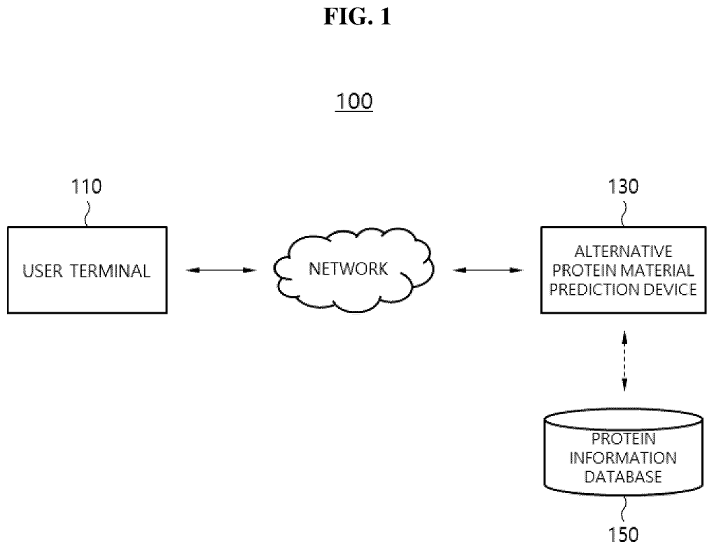

FIG. 1 is a diagram illustrating an alternative protein material prediction system according to an embodiment of the present disclosure.

Referring to FIG. 1, the alternative protein material prediction system 100 may include a user terminal 110, an alternative protein material prediction device 130, and a protein information database 150.

Although FIG. 1 illustrates a network-based alternative protein material prediction platform that may be serviced to at least one user, this is not intended to limit the scope of rights and it will be apparent to those skilled in the art that the same can be achieved using a local computing device.

The user terminal 110 may be connected to the alternative protein material prediction device 130 via a network, and may correspond to a computing terminal operated by a user that may receive recommendations for alternative protein based on the input of composite protein features in conjunction with the alternative protein material prediction device 130.

The user terminal 110 may be composed of one or more terminals. When the user terminal is composed of multiple terminals, it may include a first user terminal, a second user terminal, . . . , an nth (n is a natural number) user terminal. For example, the user terminal 110 may be implemented as a smart phone, laptop, or computer that is connected and operable with the alternative protein material prediction device 130. However, without being necessarily limited thereto, the user terminal may also be implemented as various devices including a tablet PC or the like.

Further, the user terminal 110 may access a virtual space implemented in three dimensions, such as virtual reality (VR), augmented reality (AR), or mixed reality (MR), to determine the composite features of protein, and may include a microphone module for inputting a user's voice and a display module for outputting the composite features of protein.

The alternative protein material prediction device 130 may be implemented as a server corresponding to a computer or program that performs an alternative protein material prediction service according to an embodiment of the present disclosure. The alternative protein material prediction device 130 may predict the function of protein based on various features of protein (e.g., amino acid sequence features, structural features, and physicochemical features) through protein function prediction, and may be performed using, for example, an artificial intelligence-based learning technique. The protein material prediction may play an important role in biological research, new drug development, and alternative food development. The alternative protein material prediction device 130 may be connected to the user terminal 110 via a wired network or a wireless network such as Bluetooth, WiFi, or LTE, and may transmit and receive data to and from the user terminal 110 via a wired or wireless network.

In one embodiment, the alternative protein material prediction device 130 may be implemented as a cloud server, and provide the alternative protein material prediction service to the user terminal 110 through the cloud service. In one embodiment, when the alternative protein material prediction device 130 is implemented as the cloud server, the device may provide recommendations of alternative protein to the user terminal 110 in the form of text and graphic information.

Further, the alternative protein material prediction device 130 may receive user information from the user terminal 110 and perform login. For example, the alternative protein material prediction device 130 may provide the user with the alternative protein material prediction service by receiving the user's ID and password from the user terminal 110 and performing login.

The protein information database 150 may correspond to a storage device that stores protein information for the alternative protein material prediction service.

In one embodiment, the protein information database 150 may systematically store and provide various pieces of information such as protein sequence, structure, function, interaction, expression, and mutation. To be more specific, the protein information database 150 may include information about organism, protein ID, protein sequence, gene ID, PDB (Protein Data Bank) ID, 3D structure, and protein presence. Here, the PDB (Protein Data Bank) ID is an identification code for a separate database that stores and provides the 3D structure of protein, and may obtain the structure of various biomolecules such as protein, nucleic acid, and composites.

The protein information database 150 may be used for protein sequence retrieval to obtain sequence information by searching for the name or gene name of specific protein in UniProt when one desires to know the sequence of the specific protein; protein structure analysis to search for a 3D structure of specific protein in the PDB, download atomic coordinate data, and analyze the structure using a molecular visualization tool such as PyMOL or Chimera; protein function prediction to predict the function by analyzing the protein sequence using Pfam or InterPro and identifying domains or families containing the corresponding protein; and protein-interaction network analysis to search for the interaction network of specific protein using STRING and visualize interactions with related proteins, helping to understand the protein's functional context.

In FIG. 1, the database 150 is depicted as a device independent of the alternative protein material prediction device 130. However, without being necessarily limited thereto, the database may be implemented to be included in the alternative protein material prediction device 130.

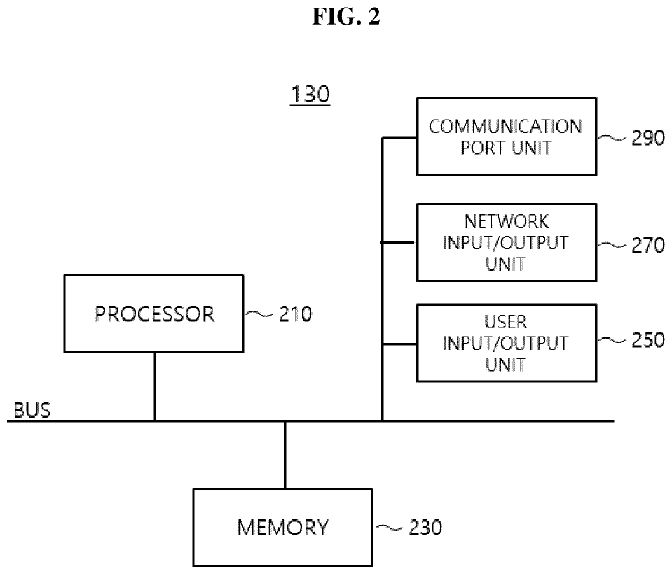

FIG. 2 is a diagram illustrating the configuration of the alternative protein material prediction device of FIG. 1.

Referring to FIG. 2, the alternative protein material prediction device 130 may include a processor 210, a memory 230, a user input/output unit 250, a network input/output unit 270, and a communication port unit 290.

The processor 210 may extract composite features of protein, generate protein graph data, and generate a report based on the execution of an alternative protein material prediction procedure as the role of predicting the alternative protein material, and may manage the memory 230 that is read or written in this process, and may schedule a synchronization time between a volatile memory and a non-volatile memory in the memory 230. The processor 210 may control the overall operation of the alternative protein material prediction device 130, and may be electrically connected to the memory 230, the user input/output unit 250, the network input/output unit 270, and the communication port unit 290 to control data flow between them. The processor 210 may be implemented as a CPU (Central Processing Unit) or GPU (Graphics Processing Unit) of the alternative protein material prediction device 130.

The memory 230 may include an auxiliary memory device that is implemented as a non-volatile memory such as an SSD (Solid State Disk) or an HDD (Hard Disk Drive) and is used to store all data required for the alternative protein material prediction device 130, and may include a main memory device implemented as a volatile memory such as a RAM (Random Access Memory). Further, the memory 230 may be executed by the electrically connected processor 210 to store a set of commands that play the role of the alternative protein material prediction device 130 according to the present disclosure.

The user input/output unit 250 may include an environment for receiving a user input and an environment for outputting specific information to the user, and may include, for example, an input device including an adapter such as a touch pad, a touch screen, an on-screen keyboard, or a pointing device, and an output device including an adapter such as a monitor or a touch screen. In one embodiment, the user input/output unit 250 may correspond to a computing device connected via remote access. In such a case, the alternative protein material prediction device 130 may operate as an independent server.

The network input/output unit 270 may provide a communication environment for connection with the user terminal 110 via the network, and may include an adapter for communication, such as, for example, a LAN (Local Area Network), a MAN (Metropolitan Area Network), a WAN (Wide Area Network), and a VAN (Value Added Network). In addition, the network input/output unit 270 may be implemented to provide a short-distance communication function such as WiFi or Bluetooth or a wireless communication function of 4G or higher for wireless transmission of data.

The communication port unit 290 is a hardware interface for connecting to external hardware. For example, the external hardware may include a printer, a mouse, or USB hardware. The communication port unit 290 may detect the connection of specific USB hardware to function as the alternative protein material prediction device 130.

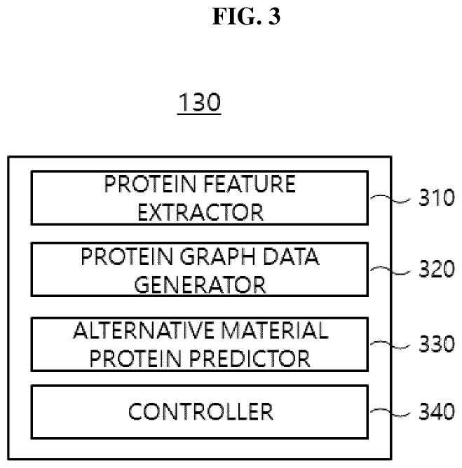

FIG. 3 is a diagram illustrating the functional configuration of the alternative protein material prediction device of FIG. 1.

Referring to FIG. 3, the alternative protein material prediction device 130 may perform the protein material prediction service of the alternative protein material prediction device 130 by extracting the composite features of protein, generating protein graph data, and performing the alternative protein material prediction procedure as the role of performing the alternative protein material prediction service according to the present disclosure, and includes a protein feature extractor 310, a protein graph data generator 320, an alternative material protein predictor 330, and a controller 340.

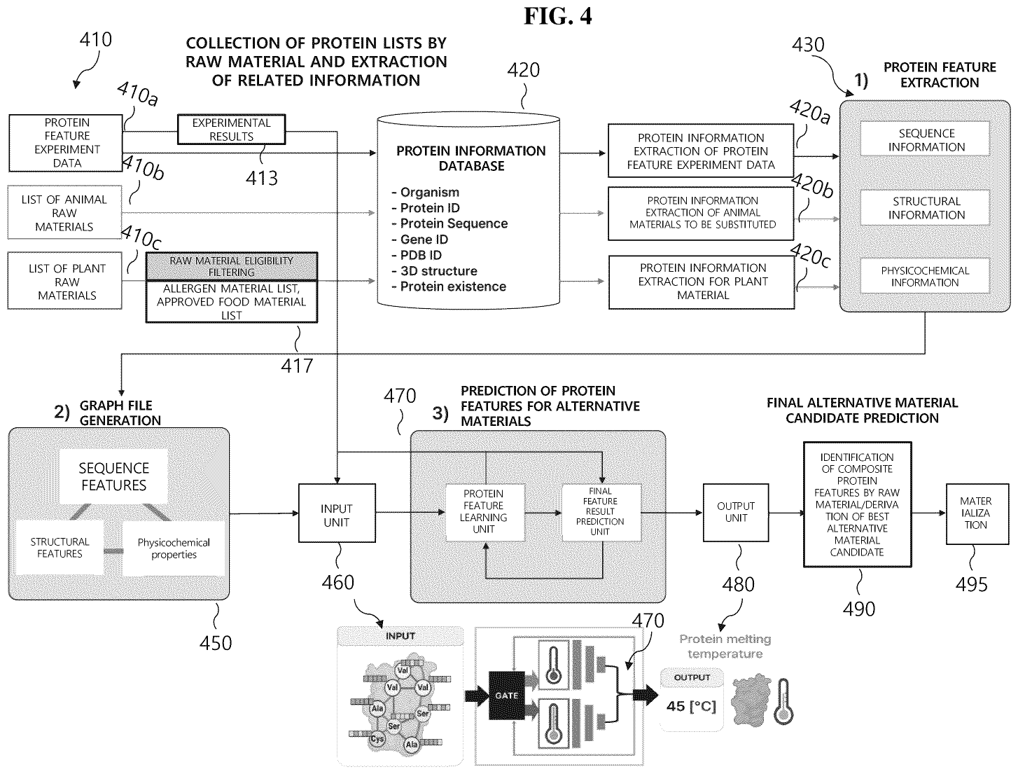

The protein feature extractor 310 extracts the composite protein features as sequence features, structural features, and physicochemical features. To be more specific, the protein feature extractor 310 may determine the sequence features, structural features, and physicochemical features of protein through the protein information database 150 that stores the composite features of animal and plant proteins. FIG. 4 is a flowchart showing the operational process of the alternative protein material prediction device 130.

The protein information database 150 (hereinafter, 420) stores a protein material 410 as protein information. That is, the protein information database may include protein feature experiment data 410a, a list 410b of animal raw materials, and a list 410c of plant raw materials as the example of the protein material 410, and may include information about organism, protein ID, protein sequence, gene ID, PDB (Protein Data Bank) ID, 3D structure, and protein presence as the example of protein information.

In one embodiment, the protein information database 420 may store protein information by pre-filtering the list 410c of plant raw materials based on their eligibility as materials, and this filtering may be performed based on an allergen material list and a list of approved food materials.

The protein feature extractor 310 may perform protein information extraction 420a of protein feature experiment data, protein information extraction 420b of animal materials to be substituted, and protein information extraction 420c for each plant material through the protein information database 420. As a result, the protein feature extractor extracts 430 the composite protein features as sequence features, structural features, and physicochemical features.

The protein feature extractor 310 may extract the sequence features of protein through a language model. In one embodiment, the protein feature extractor 310 may apply a natural language processing (NLP) technique to protein sequence analysis in the process of extracting the protein sequence features through the language model, thereby allowing the sequence features to be learned and extracted by treating the protein sequence like a character or a word.

For example, the protein feature extractor 310 may collect and preprocess protein sequence data, and may represent the sequence using a pretrained model, such as ProtTrans, and an embedding layer, after downloading a string-formatted protein sequence from the protein sequence database (e.g., UniProt and PDB). The protein feature extractor 310 may learn the language model that learns the protein sequence to extract the sequence features, and may extract the sequence features to extract the protein sequence features.

The protein feature extractor 310 extracts the physicochemical features of protein through a protein descriptor including at least CTD (Composition, Transition, Distribution) or PseAAC (Pseudo amino acid composition). For example, the protein descriptor may include CTD (Composition, Transition, Distribution), PseAAC (Pseudo amino acid composition), charge, polarity, hydrophobicity, aggregation, mass, and pl. In one embodiment, the protein feature extractor 310 may represent the amino acid composition, amino acid transition pattern, and amino acid distribution of the protein sequence through the CTD (Composition, Transition, Distribution) or PseAAC (Pseudo amino acid composition) based on the sequence information of protein. To be more specific, the composition descriptor may provide the proportion of each physicochemical group, the transition descriptor may represent a transition pattern between different groups, and the distribution descriptor may provide the position distribution of amino acid within the group. The Pseudo Amino Acid Composition (PseAAC, Pseudo AAC) is designed to better reflect various biological and chemical features of the protein sequence. Here, the PseAAC may correspond to a descriptor that numerically calculates the amino acid composition and the physicochemical features (hydrophobicity, hydrophilicity, mass, pK1, pK2, pI) of each amino acid in consideration of a sequence order. That is, the traditional amino acid composition (AAC) provides only the basic composition of amino acid in protein, whereas the PseAAC contains more pieces of information about the protein sequence. Since the order of amino acids in the protein sequence carries important information, simply using the proportion of amino acids is not sufficient. Thus, the PseAAC reflects the order information including the order information of the sequence and the physicochemical features (e.g., polarity, charge, hydrophilicity, etc.) of protein to better predict the function or structural features of protein. Further, the protein feature extractor 310 may extract physicochemical features that are important for the functional features of food protein, such as charge, polarity, hydrophobicity, secondary structure, solvent accessibility, polarizability, mass, and pl, and may analyze the unique physicochemical features of each protein.

The protein feature extractor 310 may extract structural features of protein through a graph structure data processing technique. In one embodiment, the protein feature extractor 310 represents a graph composed of nodes that express sequence features and physicochemical features among the composite features of protein, and edges that express structural features among the composite features of protein, and may learn structural features using a technique such as a Graph Neural Network (GNN). Such graph-structured data processing may be used to convert protein into a graph and train the graph neural network to learn structural features.

The protein graph data generator 320 generates the nodes based on the sequence features and physicochemical features of protein, and generates the edges between the nodes based on the structural features of protein, thereby generating protein graph data 450.

In one embodiment, the protein graph data generator 320 may assign a protein characterization code to the node based on a language representing the sequence features and amino acid feature values representing the physicochemical features.

The node may be used to express the sequence features and the physicochemical features among the composite features of protein. For example, the protein graph data generator 320 may simultaneously learn the sequence information and physicochemical features of protein by combining various properties of amino acid residues to form a node feature vector, and may generate final node features for each amino acid by combining the sequence features and the physicochemical features.

The edge may be determined based on amino-acid interaction or spatial proximity, which represent structural features, and may represent a 3D structure which represents the functional features of protein. At this time, the weight code of the edge may be determined based on the degree of the amino-acid interaction or spatial proximity. For example, the protein graph data generator 320 may generate the nodes based on the sequence features and physicochemical features of protein, and may generate the edges between the nodes based on the structural features of protein, thereby generating protein graph data.

That is, the protein graph data generator 320 may generate the protein graph data 450 based on the sequence features, the physicochemical features, and the structural features.

The alternative material protein predictor 330 learns the protein graph data 450 that reflects the composite features of protein, thereby generating an alternative protein material prediction model 470 that predicts the alternative protein material. In one embodiment, the alternative protein material prediction model 470 may be implemented as a MoE (Mixture of Expert) model. The MoE model may be composed of multiple expert networks and a gating network, and may select an appropriate expert network for each input data to improve prediction performance.

Hereinafter, the examples of using the MoE model will be described.

The protein feature extractor 310 prepares a dataset including protein sequence features, physicochemical features, and structural features, and the protein graph data generator 320 expresses the protein graph data 450 including node features and edge information.

The alternative material protein predictor 330 receives the protein graph data 450 through an input unit 460, predicts the protein features of the alternative material using the MoE model 470 that is learned with functional features data of protein verified in advance through an actual experiment, determines at least one alternative protein, and then outputs it to an output unit 480. For example, the MoE model may use a graph network neural network (GNN) that receives the protein graph data 450 as the expert network, and determine the weight of each expert through the gating network, thereby generating the alternative protein material prediction model 470. Unlike an conventional learning method that combines a classification model and a regression model, the MoE model may be trained as a single model by continuously executing the classification model and the regression model, and may be trained in a way that minimizes errors at most steps. As a result, the MoE model can solve the problems of the conventional learning method that inevitably leads to larger errors or information loss because this trains the regression model without resolving errors, and can be applied to the protein feature analysis (biodata analysis).

The alternative material protein predictor 330 receives the protein graph data 450 through the MoE model 470, determines at least one alternative protein 490 for the protein, and outputs it to the output unit 480. Further, the alternative material protein predictor 330 may recommend optimal alternative protein 495 by comparing the composite features of at least one alternative protein with the composite features of protein. That is, the alternative material protein predictor 330 may comprehensively analyze the sequence features, physicochemical features, and structural features of protein to evaluate the similarity to the original animal protein and recommend the optimal alternative protein 495.

For example, the alternative material protein predictor 330 may input the composite features of animal protein into the alternative protein material prediction model 470 to determine plant protein 490 that may substitute the animal protein.

In order to reflect the features of composite food protein, the alternative protein material prediction device 130 displays the functional features of pure single protein as well as the functional features of various proteins contained in food materials as a spectrum and comprehensively analyzes the functional features. Further, the alternative protein material prediction device 130 may analyze and compare the functional features of conventional animal protein material and the functional features of alternative material, and may provide a model that may discover the optimal alternative material through such comparison and analysis.

The controller 350 may manage the overall control operation of the alternative protein material prediction device 130, perform the protein material prediction service of the protein material prediction device 130, and manage a control flow or data flow between the protein feature extractor 310, the protein graph data generator 320, and the alternative material protein predictor 330.

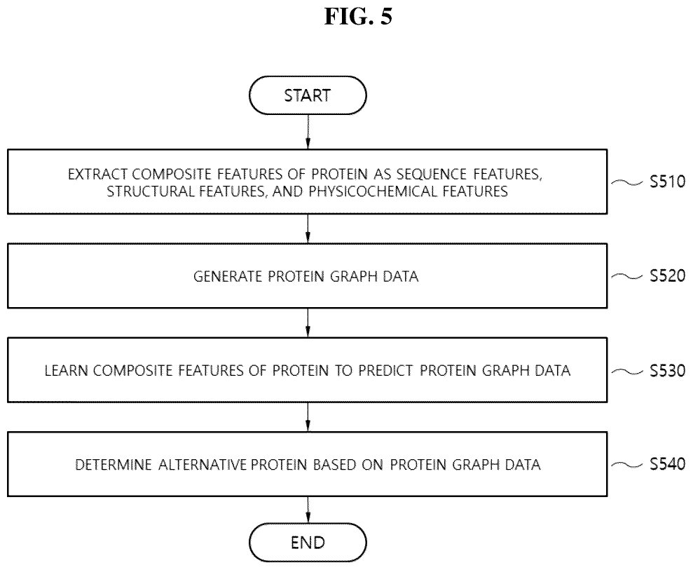

FIG. 5 is a flowchart illustrating the operation of the alternative protein material prediction device shown in FIG. 3.

In FIG. 5, the alternative protein material prediction device 130 includes a protein feature extraction step S510 that extracts the composite features of protein as sequence features, structural features, and physicochemical features, a protein graph data generation step S520 that generates protein graph data by generating nodes based on the sequence features and physicochemical features of protein and generating edges between the nodes based on the structural features of protein, and alternative material protein prediction steps S530 and S540 that generate an alternative protein material prediction model that learns the composite features of protein and predicts the protein graph data.

In step S510, the alternative protein material prediction device 130 may include a process of determining the sequence features, structural features, and physicochemical features of protein through a protein information database that stores the composite features of animal and plant proteins.

In step S520, the alternative protein material prediction device 130 may include a process of assigning a protein characterization code to the node based on a language representing the sequence features and amino acid feature values representing the physicochemical features, and assigning the weight code of the edge based on the amino-acid interaction or spatial proximity, which represent structural features.

In steps S530 and S540, the alternative protein material prediction device 130 may include a process of determining at least one alternative protein for the protein based on the predicted protein graph data.

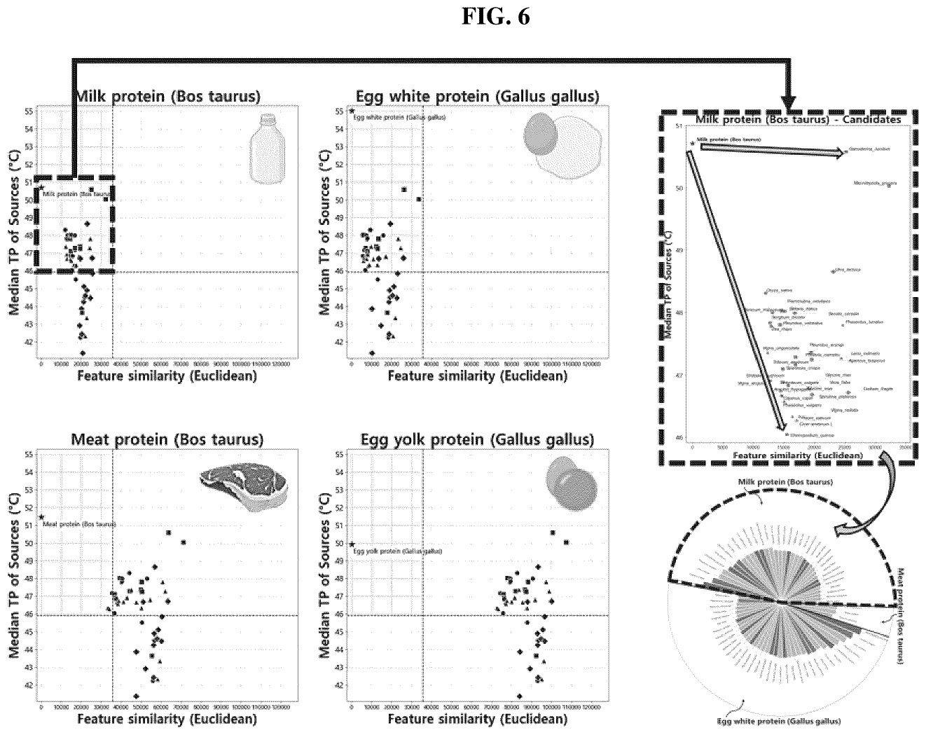

FIG. 6 is a diagram illustrating the process of selecting an alternative protein source based on the similarity of features of each raw material.

In FIG. 6, the alternative protein material prediction device 130 may perform the step of extracting features of each raw protein material and generating graph data. Here, the alternative protein material prediction device 130 may analyze the features of each raw material of plant protein to extract composite features for specific plant protein. For example, the alternative protein material prediction device 130 may receive protein sequence, amino-acid composition, and structural information from the protein information database 150, and extract the features of each raw material from the sequence and structural information. The alternative protein material prediction device 130 may convert the extracted features of each raw material into components of the protein graph data. For example, the device may assign the amino-acid sequence, physicochemical features, and thermal properties to the node of the graph, and set interaction information calculated from the 3D structure of protein as the edge of the graph, thereby generating protein graph data that reflects structural correlation between proteins.

The alternative protein material prediction device 130 may calculate a composite feature vector including information such as amino-acid composition, hydrophobicity, charge, solvent accessible surface area (SASA), surface hydrophobicity, molecular weight, aromaticity, and secondary structure ratio for each protein, and may quantitatively express the functional features of the protein through the composite feature vector. Specifically, the alternative protein material prediction device 130 may calculate the amino-acid composition that provides basic chemical composition information of protein based on the relative frequency of each amino acid that is present in the protein sequence. In addition, the alternative protein material prediction device 130 may calculate the average value, variance value, sequential pattern (transition), etc. for each amino-acid residue position or for the entire sequence based on the features such as hydrophobicity, charge, and polarity classified according to the properties of the amino acid.

Further, the alternative protein material prediction device 130 may use the 3D structural information of protein to derive structure-based features such as solvent accessible surface area (SASA), surface hydrophobicity, molecular weight, aromaticity, and secondary structure ratio (e.g., composition ratio of a-helix, B-sheet, coil, etc.). The alternative protein material prediction device 130 may generate a composite feature vector having a single fixed dimension by organizing each structure-based feature value into a numerical feature, and may also quantitatively express the structural stability, thermal characteristics, and functional similarity of the corresponding protein through the composite feature vector.

The alternative protein material prediction device 130 may perform protein feature generation and alternative candidate search steps. Here, the alternative protein material prediction device 130 may generate protein features by aggregating multiple protein feature vectors contained in the same plant raw material and determining the aggregated protein feature vectors as a representative feature vector at the unit level of the food raw material. For example, the alternative protein material prediction device 130 extracts the feature vector including sequence features, structural features, and physicochemical features for each of proteins included in the same plant raw material, normalizes the extracted feature vectors to the same dimension, and then aggregates the feature vectors to produce multiple protein feature vectors as a single vector.

In one embodiment, the alternative protein material prediction device 130 may calculate the feature similarity between the raw material features of animal protein raw materials and the raw material features of plant candidate raw materials and identify plant protein materials with high potential for substitution. For example, the alternative protein material prediction device 130 may evaluate the relative similarity between the animal protein raw material and the plant candidate raw material by applying at least one similarity measurement technique among cosine similarity, Mahalanobis distance, or function-specific scoring between the raw material features of the animal protein raw material as a reference and the raw material features of the plant candidate raw material. If the similarity value satisfies a preset reference, the device may determine that plant candidate raw material as a candidate protein source. In one embodiment, the alternative protein material prediction device 130 may perform subsequent evaluations, such as screening based on functional similarity for the derived candidate protein sources, comparison of nutritional components based on amino-acid composition, food safety analysis based on food allergy genes and toxicity genes, and candidate priority determination based on visualization.

The alternative protein material prediction device 130 may select only statistically significant properties among sequence information, physicochemical features, structural features, and features of each raw material, which constitute composite features of plant protein raw material, and use them for similarity comparison and functional analysis of alternative protein candidate groups. For example, the alternative protein material prediction device 130 may select similar candidates based on functional similarity by calculating the Euclidean distance between the features of the plant protein raw material and the protein features of the animal protein raw material (e.g., milk, egg white, meat, fish, egg yolk, etc.) as a reference. For instance, the alternative protein material prediction device 130 may visualize a location on a 2D surface based on the distance between Source Features (X-axis) and the statistical summary value (Y-axis, e.g., principal component or median, etc.) of the composite features (e.g., sequence information, physicochemical features, structural features, etc.) of each raw material, and may determine a raw material group that meets a specific threshold value (e.g., within the lower one-third of the X-axis and upper one-third of the Y-axis, etc.) as a candidate for priority screening. In addition, the alternative protein material prediction device 130 may perform distance-based ranking based on the composite feature similarity of the candidate group and the corresponding statistical values to prioritize the plant protein raw material closest to the reference animal protein.

Although the present disclosure has been described above with reference to preferred embodiments, it will understood by those skilled in the art that various modifications and changes may be made to the present disclosure without departing from the spirit and scope of the present disclosure described in the following claims.

### DETAILED DESCRIPTION OF MAIN ELEMENTS

100: alternative protein material prediction system
110: user terminal
130: alternative protein material prediction device
150: protein information database
210: processor 230: memory
250: user input/output unit 270: network input/output unit
290: communication port unit
310: protein feature extractor
320: protein graph data generator
330: alternative material protein predictor 340: controller

What is claimed is:

1. An alternative protein material prediction device comprising:
a protein feature extractor configured to extract composite features of protein as sequence features, structural features, and physicochemical features including thermal properties and functional features, or raw material-specific features;
a protein graph data generator configured to generate nodes based on the sequence features and physicochemical features of the protein and generate edges between the nodes based on the structural features of the protein, thereby generating protein graph data; and
an alternative material protein predictor configured to generate an alternative protein material prediction model for predicting an alternative protein material by learning the protein graph data that reflects the composite features of the protein.

2. The alternative protein material prediction device of claim 1, wherein the protein feature extractor determines the sequence features, structural features, and physicochemical features, and raw material-specific features of the protein through a protein information database that stores composite features of animal and plant proteins.

3. The alternative protein material prediction device of claim 1, wherein the protein feature extractor extracts the sequence features of the protein through a language model.

4. The alternative protein material prediction device of claim 1, wherein the protein feature extractor extracts the physicochemical features of the protein using a protein descriptor comprising at least CTD (Composition, Transition, Distribution) or PseAAC (Pseudo amino acid composition).

5. The alternative protein material prediction device of claim 1, wherein the protein feature extractor extracts the structural features of the protein using a graph network processing technique on the protein's graph 3D structural data.

6. The alternative protein material prediction device of claim 1, wherein the protein graph data generator assigns a protein characterization code to the node based on a language representing the sequence features and amino acid feature values representing the physicochemical features.

7. The alternative protein material prediction device of claim 6, wherein the protein graph data generator assigns weight codes of the edges based on amino acid interactions or spatial proximity that represent the structural features.

8. The alternative protein material prediction device of claim 7, wherein the protein graph data generator generates the sequence features, the physicochemical features, the structural features, and the raw material-specific features all at once as the protein graph data.

9. The alternative protein material prediction device of claim 1, wherein the alternative material protein predictor determines at least one alternative protein for the protein based on the predicted protein graph data.

10. The alternative protein material prediction device of claim 9, wherein the alternative material protein predictor compares composite features for at least one alternative protein with the composite features of the protein to recommend an optimal alternative protein.

11. The alternative protein material prediction device of claim 1, wherein the alternative material protein predictor determines plant protein capable of substituting the animal protein by inputting the composite features of animal protein into the alternative protein material prediction model.

12. The alternative protein material prediction device of claim 1, wherein the alternative material protein predictor determines a plant protein material capable of substituting the animal protein based on a functional similarity to the animal protein by inputting the composite features of the protein into a thermal property prediction model to extract raw material-specific features and calculating the Euclidean distance between the raw material-specific features.

13. An alternative protein material prediction method comprising:
a protein feature extraction step of extracting composite features of protein as sequence features, structural features, and physicochemical features;
a protein graph data generation step of generating nodes based on the sequence features and physicochemical features of the protein and generating edges between the nodes based on the structural features of the protein, thereby generating protein graph data; and
an alternative material protein prediction step of generating an alternative protein material prediction model for predicting an alternative protein material by learning the protein graph data that reflects the composite features of the protein.

14. The alternative protein material prediction method of claim 13, wherein the protein feature extraction step comprises a step of determining the sequence features, structural features, and physicochemical features of the protein through a protein information database that stores composite features of animal and plant proteins.

15. The alternative protein material prediction method of claim 13, wherein the protein graph data generation step comprises a step of assigning a protein characterization code to the node based on a language representing the sequence features and amino acid feature values representing the physicochemical features.

16. The alternative protein material prediction method of claim 13, wherein the alternative material protein prediction step comprises a step of determining at least one alternative protein for the protein based on the predicted protein graph data.

17. The alternative protein material prediction method of claim 13, wherein the alternative material protein prediction step comprises a step of determining plant protein capable of substituting the animal protein by inputting the composite features of animal protein into the alternative protein material prediction model.
